(19) United States
(12) Patent Application Publication
(10) Pub. No.: US 2024/0148920 A1
(43) Pub. Date: May 9, 2024

(54) PORTABLE DIRECT AND INDIRECT AIR AND SURFACE DISINFECTION

(71) Applicants: Kristopher Douglas Rupay, Ottawa (CA); Arash Mahinakbarzadeh, Ontario (CA); Payam Peter Bahraini, Ontario (CA)

(72) Inventors: Kristopher Douglas Rupay, Ottawa (CA); Arash Mahinakbarzadeh, Ontario (CA); Payam Peter Bahraini, Ontario (CA)

(21) Appl. No.: 17/981,911

(22) Filed: Nov. 7, 2022

(51) Int. Cl. A61L 2/10; A61L 9/20

(52) U.S. Cl. CPC A61L 2/10; A61L 9/20; A61L 2202/16; A61L 2202/25

(57) ABSTRACT

The invention is a combination of direct air and surface disinfection and air purification all in one device (three-in-one).

Disinfecting spaces can be a timely task, but with this invention surfaces and air can be disinfected in seconds, when direct disinfection is used. With humans or animals present, the device can be used in an indirect disinfection configuration to continue minimizing potential pathogens in the air.

The invention uses UVC (germicidal) lamps, a proven technology for disinfecting pathogens. When the top is off direct air and surface disinfection occurs. In this configuration, the design has been optimized by using a unique open cage design, so the light will transmit from all directions with minimal absorption to components on the device. This in combination with the lamp pattern results in a very precise UVC light emission, required for adequate, consistent disinfection of the surfaces and the air. When the top is on, the device pulls air from the lower vents, across the lamps and out the upper vents. This creates a safe, efficient indirect air disinfection system. It should be mentioned, this device is designed primarily for indirect air-disinfection. Should air-purification be required, filters can be used at the vents, capturing other (non-pathogens) in the air.

Finally, the invention consists of a real time data streaming and remote operation as data is published through a connected cloud service and then onto the subscribed users.

### BACKGROUND

Certain pathogens (specifically viruses and bacteria) have the potential to create an endemic or pandemic. These pathogens are often spread by the movement of humans throughout the world and often while in close proximity to each other. There are many places in the world where standard disinfection practices are not possible (ie. Chemicals can not be used on electronics often found in medical facilities or on board transportation vehicles). Our invention is the answer to disinfection in these places and to save time.

Our invention has been designed to quickly, efficiently, and easily inactivate the DNA and/or RNA of pathogens on surfaces and in the air, minimizing the spread. In addition, while the top is on the device it is also an indirect air disinfection and optionally an air purification system (when a filter is used).

While Ultra Violet-C light (UVC) within the Ultra Violet (UV) spectrum has been used in many other fields (medical, dental, food and beverage etc.), our design is made to bring this same technology to spaces that were not possible before.

The lamps have been placed in a configuration that allows for light to radiate from all lamps in all directions through an open cage design. This design removes much of the human error when disinfecting through more common manual methods (UVC wands or Chemical spray/wipe), that have been proven to be inconsistent.

Knowing precisely when spaces have been disinfected has become extremely valuable. The device has been designed to provide a wealth of data throughout the operation. The device has both Wi-Fi and cellular connection ensuring it remains connected throughout its use and while charging. The data on board the invention is streamed in real-time providing valuable information that can be shared amongst operators and other subscribed users.

### SUMMARY

This invention was designed to disinfect the air and high touch surfaces of spaces in two different methodologies. Although, such devices exist that provide either methodology, the focus of this invention is to combine indirect air disinfection, air purification, direct air disinfection and direct surface disinfection. The device has been designed to be portable, designed to reduce disinfection time, increase disinfection consistency, and provide real-time data that can be used for disinfection record keeping.

Distinguishing features of the invention include:
1) Efficient Direct Air and Surface Disinfection: A unique UVC lamp and open cage configuration has been designed to minimize light absorption, maximize light emission, radiate in all directions, and require zero mechanical adjustments and/or human intervention while in operation.
2) Air Purification: The device can be placed into an indirect air disinfection and air purification configuration that is safe for humans and animals to be in close proximity.
3) Portability: Dimensions, weight and handle placements have been uniquely designed to be light weight, portable and easily fit into tight, spaces.
4) Connectivity: The device remains connected (both Wi-Fi and cellular) prior, during and after operation, providing real time data for statistics, status, remote dashboards, and maintenance. The connection enables, remote operation through a personal computer, laptop, tablet, or mobile device as a secondary option for control.

### BRIEF DESCRIPTION OF DRAWINGS

FIG. 1: A view with the device in direct air and surface disinfection mode.

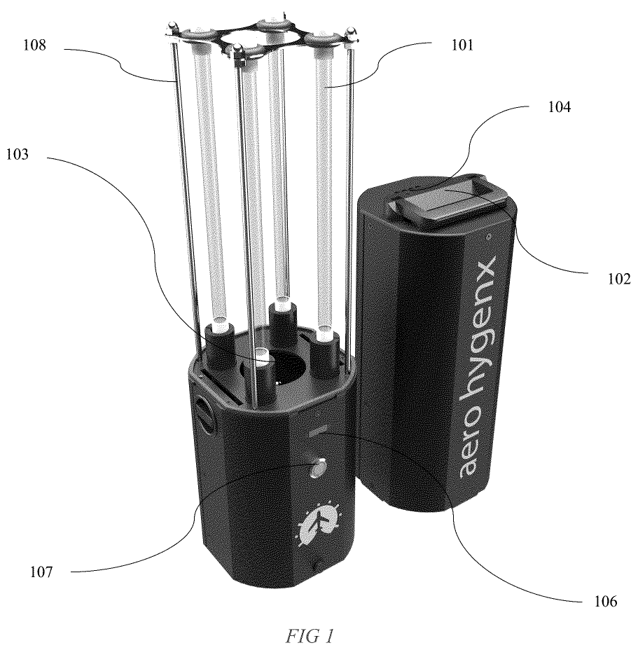

FIG. 2: A view of the device in indirect air disinfection and air purification mode.

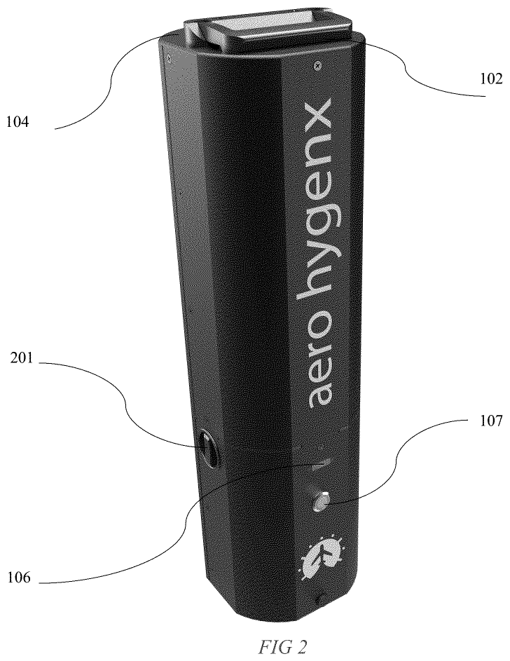

FIG. 3: A view of the device with the lamps powered on in direct air and surface disinfection mode.

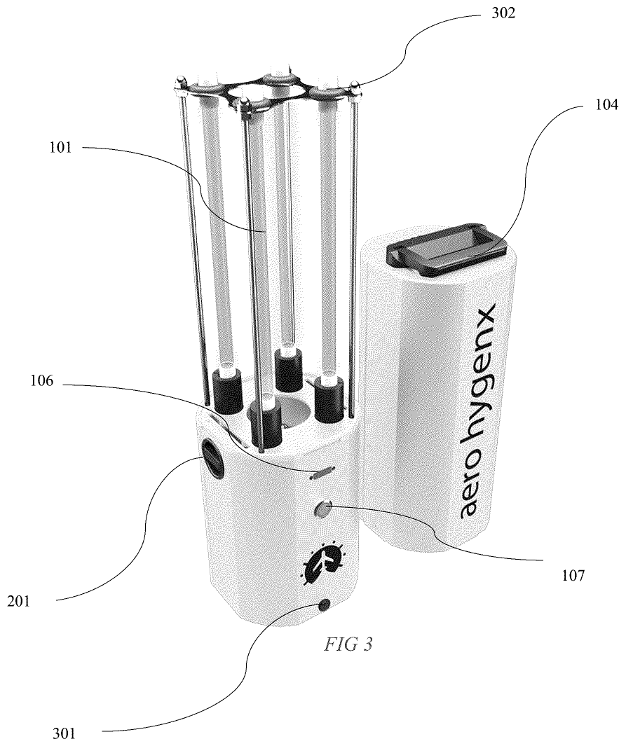

### DETAILED DESCRIPTION

Our invention brings together many technologies to ensure the disinfection of tight spaces. The invention focuses on efficient direct surface and air UV disinfection, Air disinfection, Air purification, device portability, and connectivity.

The disinfection technology that is being utilized is germicidal UVC electromagnetic radiation lamps. Germicidal lamps 101 produce a unique wavelength of light, know to scientists for over a century. It yields incredibly high disinfection results when the correct dosage of light reaches a surface or passes through open space. Wavelengths in this spectrum of light transmit right to the pathogens DNA or RNA. When the light reaches the DNA or RNA it alters this genetic material, preventing these cells from ever replicating again, thus stopping the spread of the pathogen.

UVC is known to have a very low reflectivity off nearly all surfaces. The invention has been designed without any vertical reflective components (often found in the centre of UV emitting devices, between lamps), utilizing an open cage design. The germicidal lamps 101 radiate light in all directions around the cylinder of every lamp (360 degrees), and in all directions up and down (180 degrees) along the length of the lamp. The open cage design enables minimal light absorption, thus allowing the maximum amount of light to radiate away from the device. By maximizing the radiation of light, the design can now reduce the overall footprint of the design and reduce the required batteries to power the device.

Nearly every component on the device is made of aluminium for light weight (again aiding to portability). Aluminum is a softer metal than most, thus strategic single, double, and even triple bends are used to ensure the required strength requirements were met.

A single battery design is used and placed at the lowest point of the device. As the heaviest component on the device, this was intended to ensure a very low centre of gravity on the device. The battery selected is a trade secret, but it is a battery known to have light weight properties, operate in a very wide range of temperatures (−40° C. to +70° C.), have a consistent voltage throughout operation, is safe to travel with, can quickly be charged and has an incredibly long-life span (more than 2000 full cycles).

To start the device a single button 107 can be found on the front 204. The button will then start to flash, warning operators that disinfection is about to occur. The time for the countdown is configurable and can be configured through the app or by scanning QR codes containing information about the space dimensions. Should any errors with the device be detected, a glowing light on the start button 107 will not be illuminated.

When running a device such as this, safety is a major part of the autonomous design. The design includes infra-red sensors 106 to detect human movement within close proximity.

In addition to the automated safety features, manual safety features have been included within the design. The start button 107 can be pressed at any given time and will immediately stop the disinfection.

Connectivity and the data that is exchanged is a major part of our invention design. The device has been designed to communicate primarily through wireless communication, but it also has a cellular connection should wireless communication not be available. Trade marked the “HygenX Stream”, this connection enables the following:
1) Real Time Data Exchange.
2) Remote Control of device.
3) Remote Health Monitoring.
4) Remote software updates.
5) Remote adjustments for custom movement scenarios.
6) Secure shell communication.

In the world of IoT, it is paramount that the design includes an API to securely share real time data from the devices. The robotic and light-based data can be accessed “raw” for health monitoring or is turned into essential statistical based data for subscribed users. This data includes, but is not limited to:
1) Disinfection Status (Percentage complete)
2) Lifetime usage.
3) Total Disinfections.
4) Total Disinfection Time.
5) Disinfections per day.
6) Predictive maintenance (i.e., Expecting a lamp degradation).
7) Time savings by using device compared to other devices.

This information, provided through the Hygenx Stream API, is intended for operators where time is critical or to passengers/transit riders looking for confidence that their mass transportation vehicle has been disinfected to the intended level. This data is also collected over a configurable period of time (configured from app), where disinfection reports can be generated showing exactly when each space and for how long was disinfected.

The HygenX Stream allows operators to control their device(s) from a central control centre or personal device (personal computer, tablet, or mobile device). Operators can view all of their devices, select the device of interest and control with all of the same buttons that are located on the local physical touch of the device.

Support and maintenance are paramount to time critical operations, thus having the ability to update software, monitor system status and remote directly to the device (using secure shell) is essential. The HygenX Stream comes equipped with all of this.

With reference to the figures, the following table describes the key hardware components within the invention.

| | Name | Description |
|---|---|---|
| 101 | Lamps | UVC lamps that emit the correct wavelength (~254 nm) of light to |
| | | destroy pathogens. The lamps have been placed in an open cage |
| | | design to minimize absorption of light. |
| 102 | Handle | Ergonomic handle, used to carry the device. |
| 103 | Air purification | When the top of the device is in place, air passes upwards through |
| | middle vent | this channel the across the lamps. |
| 104 | Air purification | Cleaned (disinfected) air exits this vents after passing in front of the |
| | upper vents | UVC light. |
| 106 | IR Sensor | One of two IR sensors on board the device, to detect human motion. |
| 107 | Master Switch | This button will power on the device, start the device, communicate |
| | | system health, provide a countdown and power down the device. |
| 108 | Lamp Protection | These rods are used to protect the lamps. The narrow rods are used |
| | Rods | for support while encouraging the open cage design. |
| 201 | Lock for Lid | When the lid is in place the locks will line up, to ensure safe |
| | | operation. |
| 301 | Thumb Screws | Quick access for battery swap. A tool-free thumb screw can be |
| | | used on either side. |
| 302 | Rubber Grommets | Used to protect the upper ceramics of the lamps. |

### Alternate Embodiments

As the technology evolves, we are considering the utilization of alternate UVC producing technologies. UV-LEDs are growing in popularity, as they are more robust (typically producing light around 280 nm), unfortunately their efficiency is not great enough for this application to be possible at the time of this writing. Lamps producing UVC light less than 230 nm is of interest, as research is showing it may be safe for prolonged exposure to human skin or eyes. Similar to LEDs, the efficiency and cost is not nearly as good as the traditional 254 nm lamps at this time.

### REFERENCES

Bolton, J. R., 2010. “Ultraviolet Applications Handbook”, 3rd, ICC Lifelong Learn Inc., 628 Cheriton Cres., NW, Edmonton, AB, Canada T6R 2M5.

The invention claimed is:

1. A portable direct UVC disinfection device designed to quickly disinfect spaces where either chemicals can not be used, where chemicals can not kill the specific strain of virus, where disinfection consistency is required and when short disinfection times are required.

2. A portable UVC indirect air disinfection and air purification device that can be used to disinfect the air in rooms while humans and/or animals are present.

3. Connectivity: The device remains connected (including Wi-Fi, cellular and Bluetooth) prior, during and after operation, providing real time data for statistics, status, remote dashboards, and maintenance; and remote operation through a personal computer, laptop, tablet, or mobile device as a secondary option for control.
